(19) United States
(12) Patent Application Publication
(10) Pub. No.: US 2009/0081694 A1
(43) Pub. Date: Mar. 26, 2009

(54) Modified well plates for molecular binding studies

(75) Inventors: John Lawrence Ervin, San Diego, CA (US); Hus Tigli, La Jolla, CA (US); Mark R. Kennedy, South Burlington, VT (US)

Correspondence Address: TREX ENTERPRISES CORP., 10455 PACIFIC COURT, SAN DIEGO, CA, 92121, US

(73) Assignee: Trex Enterprises Corp.

(21) Appl. No.: 12/221,140

(22) Filed: Jul. 30, 2008

Related U.S. Application Data
(63) Continuation-in-part of application No. 11/180,349, filed on Jul. 13, 2005.
Application No. 11/180,349 is a continuation-in-part of application No. 10/631,592, filed on Jul. 30, 2003.
Application No. 10/631,592 is a continuation-in-part of application No. 10/616,251, filed on Jul. 8, 2003.
(60) Provisional application No. 60/962,652, filed on Jul. 30, 2007.
(60) Provisional application No. 60/962,616, filed on Jul. 30, 2007.
(60) Provisional application No. 60/962,664, filed on Jul. 30, 2007.
(60) Provisional application No. 60/962,756, filed on Jul. 30, 2007.
(60) Provisional application No. 60/962,675, filed on Jul. 30, 2007.
(60) Provisional application No. 60/962,669, filed on Jul. 30, 2007.
(60) Provisional application No. 60/962,644, filed on Jul. 30, 2007.
(60) Provisional application No. 61/127,910, filed on May 15, 2008.

(51) Int. Cl. G01N 33/53

(52) U.S. Cl. 435/7.1

(57) ABSTRACT

Microtiter plates modified to permit their use for molecular binding studies.

### CROSS-REFERENCE TO RELATED APPLICATIONS

This application claims the benefit of provisional patent application Ser. Nos. 60/962,652, 60/962,616, 60/962,664, 60/962,756, 60/962,675, 60/962,669 and 60/962,644 all filed Jul. 30, 2007 and provisional patent application Ser. No. 61/127,910, filed May 15, 2008 and is a continuation in part of Ser. No. 11/180,349 filed Jul. 13, 2005, Ser. No. 10/631,592 filed Jul. 30, 2003 and Ser. No. 10/616,251 filed Jul. 8, 2003.

### FIELD OF INVENTION

This invention relates to devices for monitoring molecular binding interactions and in particular to such devices utilizing micro-titer plates

### BACKGROUND OF THE INVENTION

### Optical Biosensors

An optical biosensor is an optical sensor that incorporates a biological sensing element. In recent years optical biosensors have become widely used for sensitive molecular binding measurements. To study interactions of proteins with other biomolecules one may generally use labeled or label-free methods. For these methods a first molecule of interest (the receptor) is immobilized onto a surface. An interaction is monitored by then introducing additional molecules (the targets) and detecting whether they in fact bind to the receptor. When using labels to monitor these interactions a fluorescent, colorimetric or some other signal is generated by an additional molecule or moiety that is attached to the target or receptor which gives a signal when the interaction takes place. This so called label (or tag) is present only to detect the interaction and is not part of the interaction of interest per se.

In label free binding, on the other hand, the receptor and target binding are monitored directly using untagged biomolecules. A variety of technologies exist in the art to detect binding without labels including surface plasmon resonance (SPR) and white light interferometry using porous silicon. In addition to the variety of technologies which exist to monitor label free binding events, there are a variety of instrument architectures which can used. These include plate readers and flow cells. In the case of plate readers a well plate (or micro well plate or micro titer plate) is used to house the biochips and fluids which are used for the label free binding studies. This allows for parallel analyses of several types of data. Alternatively flow cells house biochips in, typically, a microfluidic cell which routes fluid over the region of the biochip where the binding interaction takes place.

When acquiring and analyzing data of this sort there are a number of steps which are performed for the data analysis (the data method) on a number of channels (be those channels, flow cells or wells in a well plate). A file format which captures the full gamut of what a user of the analytical instrument might want to do must incorporate flexibility in acquisition and in analysis.

### Surface Plasmon Resonance

An optical biosensor technique that has gained increasing importance over the last decade is the surface plasmon resonance (SPR) technique. This technique involves the measurement of light reflected into a narrow range of angles from a front side of a very thin metal film producing changes in an evanescent wave that penetrates the metal film. Ligands and analytes are located in the region of the evanescent wave on the backside of the metal film. Binding and disassociation actions between the ligands and analytes can be measured by monitoring the reflected light in real time. These SPR sensors are typically very expensive. As a result, the technique is impractical for many applications.

### Resonant Mirror

Another optical biosensor is known as a resonant mirror system, also relies on changes in a penetrating evanescent wave. This system is similar to SPR and, like it, binding reactions between receptors and analytes in a region extremely close to the back side of a special mirror (referred to as a resonant mirror) can be analyzed by examining light reflected when a laser beam directed at the mirror is repeatedly swept through an arc of specific angles. Like SPR sensors, resonant mirror systems are expensive and impractical for many applications.

### Thin Films

It is well known that monochromic light from a point source reflected from both surfaces of a film only a few wavelengths thick produces interference fringes and that white light reflected from a point source produces spectral patterns that depend on the direction of the incident light and the index of refraction of film material. (See “Optics” by Eugene Hecht and Alfred Zajac, pg. 295-309, Addison-Wesley, 1979.)

### Porous Silicon Layers

U.S. Pat. No. 6,248,539 (incorporated herein by reference) discloses techniques for making porous silicon and an optical resonance technique that utilizes a very thin porous silicon layer within which binding reactions between ligands and analytes take place. The association and disassociation of molecular interactions affects the index of refraction within the thin porous silicon layer. Light reflected from the thin film produces interference patterns that can be monitored with a CCD detector array. The extent of binding can be determined from change in the spectral pattern.

### Kinetic Binding Measurements

Kinetic binding measurements involve the measurement of rates of association (molecular binding) and disassociation. Analyte molecules are introduced to ligand molecules producing binding and disassociation interactions between the analyte molecules and the ligand molecules. Association occurs at a characteristic rate [A][B]kₒₙ that depends on the strength of the binding interaction kₒₙ and the ligand topologies, as well as the concentrations [A] and [B] of the analyte molecules A and ligand molecules B, respectively. Binding events are usually followed by a disassociation event, occurring at a characteristic rate [A][B]k_{off} that also depends on the strength of the binding interaction. Measurements of rate constants kₒₙ and k_{off} for specific molecular interactions are important for understanding detailed structures and functions of protein molecules. In addition to the optical biosensors discussed above, scientists perform kinetic binding measurements using other separations methods on solid surfaces combined with expensive detection methods (such as capillary liquid chromatography/mass spectrometry) or solution-phase assays. These methods suffer from disadvantages of cost, the need for expertise, imprecision and other factors.

### Separations-Based Measurements

More recently, optical biosensors have been used as an alternative to conventional separations-based instrumentation and other methods. Most separations-based techniques have typically included 1) liquid chromatography, flow-through techniques involving immobilization of capture molecules on packed beads that allow for the separation of target molecules from a solution and subsequent elution under different chemical or other conditions to enable detection; 2) electrophoresis, a separations technique in which molecules are detected based on their charge-to-mass ratio; and 3) immunoassays, separations based on the immune response of antigens to antibodies. These separations methods involve a variety of detection techniques, including ultraviolet absorbance, fluorescence and even mass spectrometry. The format also lends itself to measure of concentration and for non-quantitative on/off detection assays.

To study interactions of proteins with other biomolecules one may generally use labeled or label-free methods. For these methods a first molecule of interest (the receptor) is immobilized onto a surface. An interaction is monitored by then introducing additional molecules (the targets) and detecting whether they in fact bind to the receptor. When using labels to monitor these interactions a fluorescent, colorimetric or some other signal is generated by an additional molecule or moiety that is attached to the target or receptor which gives a signal when the interaction takes place. This so called label (or tag) is present only to detect the interaction and is not part of the interaction of interest per se. In label free binding, on the other hand, the receptor and target binding is monitored directly using untagged biomolecules.

### Micro-Titer Plates

The microtiter plate (sometimes wellplate or microwellplate) is a ca. 5.0×3.4″ plastic plate which contains fluid chambers or wells used for chemical and biochemical research. This well known plate format has a variety of standard well placements including a ‘96-well plate’ and a ‘384-well plate’. Given the wide use of these wellplates in research modifying these well plates to accommodate label free binding biochips is important.

An appropriate apparatus for adapting the standard well plate format would allow an instrument to optically read the plate as well as allow an automated liquid handler to add and remove liquids to the biochips. A proper system should also allow for the setting of time=0 during the initiation of either a binding or an unbinding event.

What is needed are microtiter plates modified to permit their use for label-free molecular binding studies.

### SUMMARY OF THE INVENTION

The present invention provides microtiter plates modified to permit their use for molecular binding studies.

### BRIEF DESCRIPTION OF THE DRAWINGS

FIG. 1 shows a technique for holding porous silicon chips in a plastic 8-position manifold.

FIG. 2 sketch of a single well containing the porous silicon chip in a plastic 8-pos manifold

FIG. 3 shows a technique for converting a 96-well micro-plate for flow cell measurements.

FIG. 4 shows a cross section of a well of a micro-well plate with a porous silicon chip in place for flow cell measurements.

FIG. 5 shows results from well plate data A is anti-IgG molecule bound to the chips in a well strip B is the result of immobilizing IgG to the aIgG prepared chips

FIG. 6 shows the results from FIG. 5 shown on an affinity graph in which the dissociation equilibrium constant of the interaction may be calculated.

### DETAILED DESCRIPTION OF PREFERRED EMBODIMENTS

A preferred embodiment of the present invention is embedded microprocessor based controller that is programmable in a graphical language for controlling designed for driving an optical biosensor described in parent patent application Ser. No. 11/180,349 filed Jul. 13, 2005, Ser. No. 10/631,592 filed Jul. 30, 2003 and Ser. No. 10/616,251 filed Jul. 8, 2003 and Ser. No. ______ entitled “Optical Sensor and Methods for Measuring Molecular Binding Interactions” which is being filed simultaneously with this application. All of the above applications are incorporated herein by reference.

### Example 1

In this embodiment, 3.5 mm square porous silicon biosensor chips are glued or otherwise affixed to the bottom of a 96 well plate. Here an optical probe is scanned across the top of the well plate. Biochips are illuminated from the top and reflected light is captured in the same probe. Here the chip's optically active region is facing upwards.

In this example the initiation of a binding or unbinding step is set by the addition of fluid to the system.

### Example 2

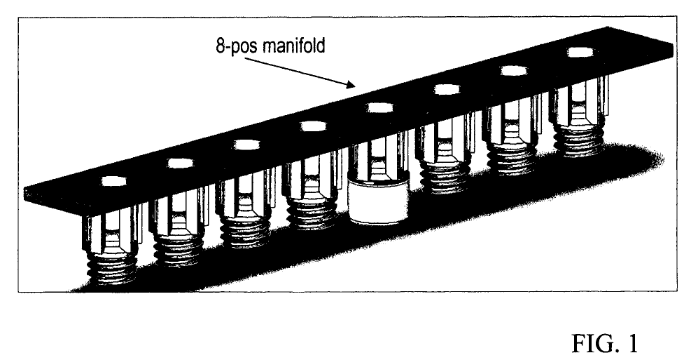

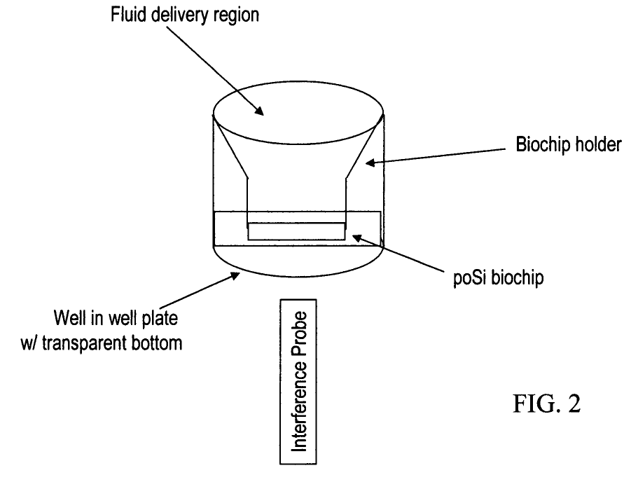

In this embodiment (FIG. 1), 2.6 mm square porous silicon biosensor chips are held in a plastic 8-position manifold to form a well strip. Here, the chip's optically active region is facing downwards (FIG. 2). In this case the light to and from the optical probe comes from the bottom of the well plate passing though its transparent bottom. It then passes through the fluid containing the biomolecules then through a hole in the cap where it interferes with the porous silicon layer and is reflected back to the optical probe.

In this example the initiation of a binding or unbinding step is set by the well strip being plunged into a set of eight wells that have been loaded with the appropriate fluid for that step. This plunging may be done by hand or may be done using a robotic system. Also, the read out of the chips may be done one at a time in groups larger than one depending on how many optical channels are present in the instrument to read them out.

### Example 3

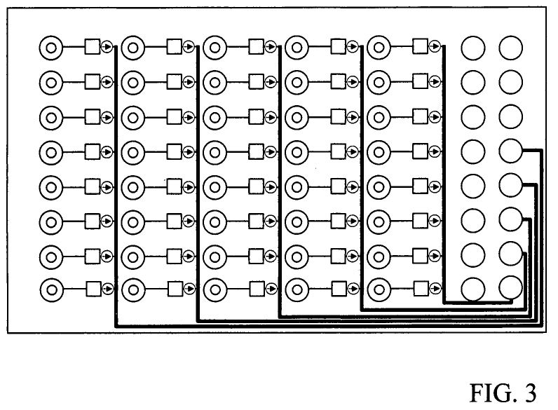

In this embodiment (FIG. 3), 3.5 mm square porous silicon biosensor chips are embedded in the actual microplate itself. Here the first column is used as fluidic ports whose injected fluid is routed by embedded microfluidics across chips embedded in the second column. The fluid then goes through a check valve (formed for instance by a fluidic restriction) and onto a well dedicated to waste in the last column of the well plate. Columns repeat like this with a column for fluidic ports and a column for bio chips until the last two columns of the well plate which are reserved for reagents or waste.

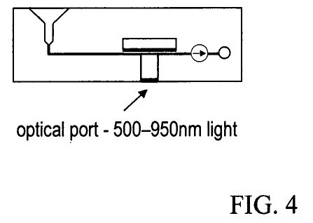

The well plate is constructed so that there are optically addressable areas on the bottom for the optical readout system (FIG. 4). The biochip is shown suspended upside down in the well plate itself. The microfluidics route the fluid from the fluidic port in the left hand well position onto the biochip itself through a check valve formed by introducing a restriction into the microfluidics. The fluid then goes into a channel of much lower impedance to waste.

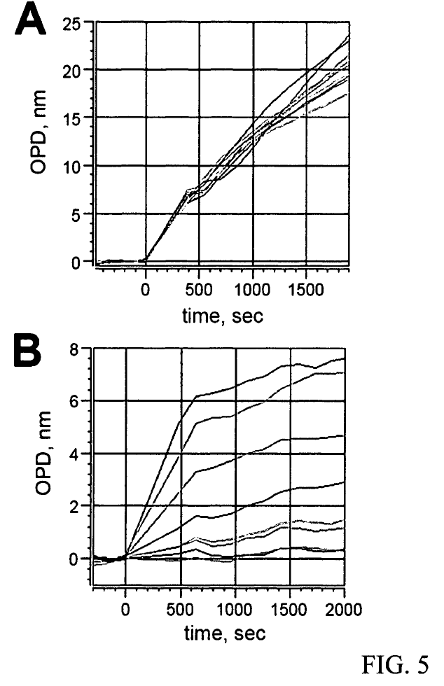

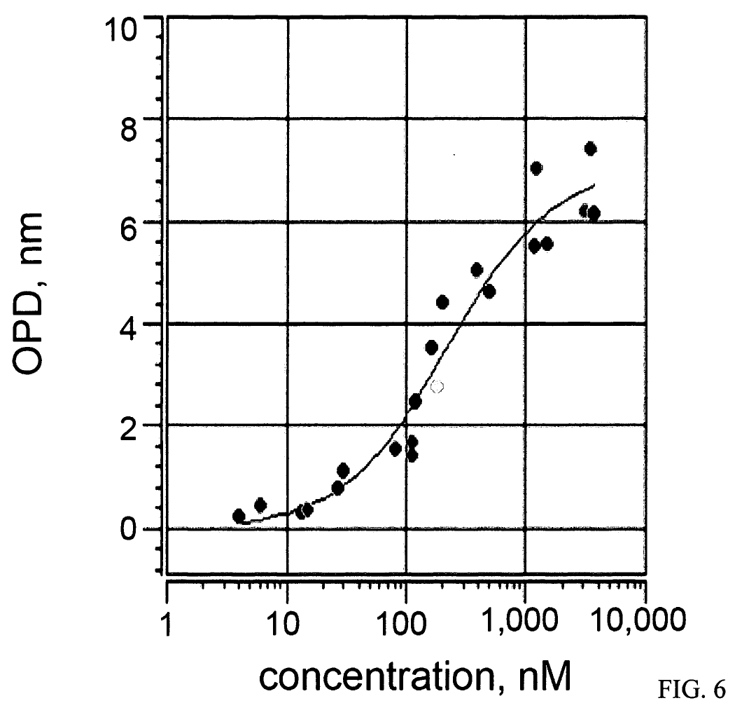

These modified microtiter plates may then be used for a variety of label-free binding experiments. Shown in FIG. 5 are the results from a typical study using example 2. A protein antibody molecule, anti-IgG, is covalently immobilized to poSi chips in a microtiter plate (FIG. 5 A) and the amount of immobilization monitored in real time. In FIG. 5 B several concentrations of human IgG are then introduced into the several chips. These real time traces are then analyzed by plotting the equilibrium binding amount against the concentration (FIG. 6). A fit of this graph to a two-state model then gives the dissociation equilibrium constant.

What is claimed is:

1. A method for modifying standard microtiter plates to allow their use in label free binding studies by coupling the microtiter plates with porous silicon biosensor chips.

2. The method as in claim 1 wherein the biochips are affixed to the bottom of the well plate.

3. The method as in claim 2 wherein the biochips are made using porous silicon.

4. The method as in claim 1 wherein the biochips are held in the well plate using a holder.

5. The method as in claim 4 wherein the holder holds several biochips.

6. The method as in claim 4 wherein the biochips are made using porous silicon.

7. The method as in claim 1 wherein the biochips are directly incorporated into the well plate itself.

8. The method as in claim 7 wherein the biochips are made using porous silicon.

9. The method as in claim 1 wherein the biochips are monitored using optical interferometry.

10. A method for using biochips to study time resolved biomolecular interactions comprising:
A) Holding the biochips in an injection molded carrier.
B) Setting time=0 by plunging the carrier and biochip into the prepared the wells of a microtiter plate.

11. The method as in claim 4 wherein the well plate has a transparent bottom suitable to reading out the biosensor chips from the bottom.
